(19) United States
(12) Patent Application Publication
(10) Pub. No.: US 2004/0138182 A1
(43) Pub. Date: Jul. 15, 2004

(54) Colchicine derivatives, process for preparing them, products obtained therefrom and use thereof

(75) Inventors: Christian Wehrey, Villemomble (FR); Serge Droux, Dammartin En Goele (FR)

Correspondence Address: ROSS J. OEHLER, AVENTIS PHARMACEUTICALS INC., ROUTE 202-206, MAIL CODE: D303A, BRIDGEWATER, NJ, 08807, US

(73) Assignee: AVENTIS PHARMA S.A., Antony (FR)

(21) Appl. No.: 10/731,842

(22) Filed: Dec. 5, 2003

(30) Foreign Application Priority Data
Dec. 6, 2002 (FR) 0215418

(51) Int. Cl. A61K 31/66; C07F 9/02

(52) U.S. Cl. 514/114; 558/110

(57) ABSTRACT

This invention discloses and claims processes for preparing colchicine derivatives, products obtained by these processes, and use thereof. More specifically, the invention relates essentially to a process for preparing organophosphorus compounds and their salts, having therapeutic activity, in particular in oncology.

This application claims the benefit of priority of French Patent Application No. 02/15,418, filed Dec. 6, 2002.

The present invention relates generally, and according to a first of its aspects, to a novel process for preparing colchicine derivatives.

More particularly, the present invention relates to a process for preparing products of general formula 1:

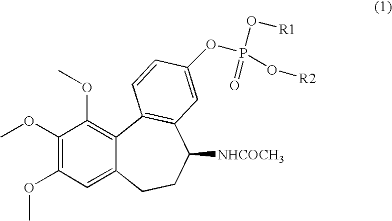

The products of general formula 1 are derivatives of colchicine and of colchiceine. Colchicine and coichiceine are natural alkaloids extracted from Colchicum autumnale, a plant of the family Liliaceae. Colchicine is known for its anti-mitotic properties and its ability to bind to tubulin (J. M. Andreu, S. N. Timasheff, Proc. Nat. Acad. Sci. USA 79, 6753, (1982).

Many derivatives of colchicine and of colchiceine have been prepared to date. For instance, Patent Applications WO 99/02166, WO 00/04434 and WO 00/40529 disclose and claim colchicine derivatives.

These patent applications describe products of general formula 1, in which the substituents R1 and R2 are carbonaceous radicals selected for being able to be prepared by means of radical-generating oxidizing compounds, for example meta-chloroperbenzoic acid (MCPBA), and then to be cleaved with trifluoroacetic acid (TFA), so as to obtain a phosphoric acid of general formula 1, in which R1=R2=H. This product is called colchinol phosphate, without the counter-ions linked to the phosphate being considered.

However, the use of MCPBA poses certain problems, associated, firstly, with its relative instability and, secondly, with the difficulty in isolating and purifying the expected products. Consequently, such a process poses problems in terms of industrialization.

In addition, the colchinol phosphates are especially sensitive to pH conditions. Thus, an acceptable preparation process will have to use gentle reaction and treatment conditions, otherwise risking cleavage of the phosphate group and/or racemization of the product.

George R. Pettit et al., Anti-Cancer Drug Design (1995), 10, 299-309, disclose processes for preparing combretastatin derivatives, in particular phosphates. Two methods for preparing these products are presented (pp. 304-306). The protected phosphate group is condensed with the phenol of the combretastatin by reaction in pyridine at 25° C. for 15 h and then at 90° C. for 2.5 hours in the case of the first method, and at 60° C. for 10 hours then at ambient temperature for 56 hours in the case of the second method.

Use of the methods described in Anti-Cancer Drug Design (1995), 10, 299-309 has not made it possible to obtain satisfactory results: in all cases, the reaction is very slow and the yield is very low at approximately 20% of expected product. Attempts at successive additions of phosphorus-containing reagent have not made it possible to improve this yield. Moreover; isolation of the product is difficult.

Surprisingly, and against all expectations, it has been found that it is possible to obtain results which are clearly better in terms of yield of condensation of the phosphate group with the colchinol by substituting the pyridine with a compound containing a nonaromatic amine function.

Among the nonaromatic amines which can be envisaged, trialkylamines are preferred. A more preferred trialkylamine is triethylamine.

One of the advantages of the invention is also to make it possible to perform the entire coupling reaction at ambient temperature, without having to heat it, as is the case for the process described by George R. Pettit et al.

Another of the advantages of the invention is to make it possible to readily isolate the product obtained by extraction using conventional techniques readily adaptable to the production of large amounts of the product.

According to a first aspect, the invention relates to a process for preparing a product of general formula 1:

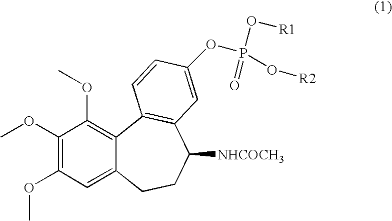

comprising a step consisting of coupling between a compound of general formula 2:

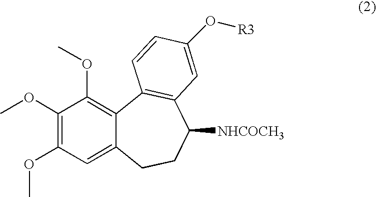

and a compound of general formula 3:

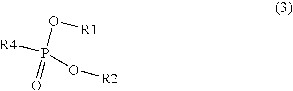

in which

(i) R1 and R2 are independently selected from the group consisting of alkyl, cycloalkyl, substituted alkyl and substituted cycloalkyl,

(ii) or else R1 and R2 together form a single substituent chosen from alkyl, cycloalkyl, substituted alkyl and substituted cycloalkyl, and

(iii) R3 and R4 are labile substituents,

in the presence of a compound comprising a nonaromatic amine function.

A preferred compound comprising a nonaromatic amine function is a trialkylamine, preferably triethylamine.

The reaction is advantageously carried out in the presence of a halogenated solvent.

A preferred halogenated solvent is dichloromethane.

R1 and R2 are advantageously halogenated aliphatic groups or together form a single halogenated aliphatic group.

An acceptable halogenated aliphatic group may be chosen from carbonaceous chains substituted with at least one halogen selected from the group consisting of chlorine, bromine and iodine.

The carbonaceous chain will advantageously comprise a perhalogenated free terminal portion, preferably having a unit of the —CH₂—R_{Cl} type, R_{Cl} being a perchlorinated residue.

More preferably, R1 and R2 may each be a 2,2,2-trichloroethyl substituent.

R3 is advantageously chosen from H, Li, Na and K. A more preferred substituent R3 is H.

R4 is advantageously chosen from Cl, Br and I. A more preferred substituent R4 is Cl.

A process in accordance with the invention may be used particularly advantageously when the compound of general formula 1 is bis(2,2,2-tri-chloroethyl) (5S)-5-acetylamino-9,10,11-trimethoxy-6,7-dihydro-5H-dibenzo-[a,c]cyclohepten-3-ylphosphate.

A process in accordance with the invention may be used particularly advantageously when the compound of general formula 3 is N-[(5S)-3-hydroxy-9,10,11 -trimethoxy-6,7-dihydro-5H-dibenzo[a,c]cyclohepten-5-yl]-acetamide, and the compound of general formula 4 is bis(2,2,2-trichloroethyl) phosphorylchloride.

The coupling reaction between the compound of general formula 3 and the compound of general formula 4 is preferably carried out between 0 and 100° C., more preferably between 20 and 100° C., very preferably between 20 and 50° C.

According to a second aspect, the invention relates to the products obtained according to its first aspect.

According to a third aspect, the invention relates to a process for preparing a compound of formula 4:

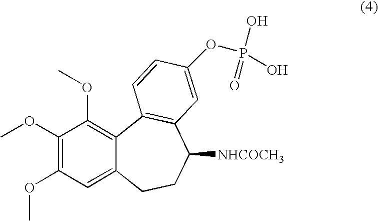

comprising a step in which a product according to its second aspect undergoes cleavage of the substituents R1 and R2 in the presence of at least one transition metal, preferably zinc.

The substituents R1 and R2 are cleaved more advantageously in the presence of two different transition metals, preferably zinc and copper.

The compound of formula 4 may also be purified by passing it over ion exchange resin.

According to a fifth aspect, the invention relates to the products obtained by a process in accordance with its fourth aspect.

According to a sixth aspect, the invention relates to a process for preparing a compound of general formula 5:

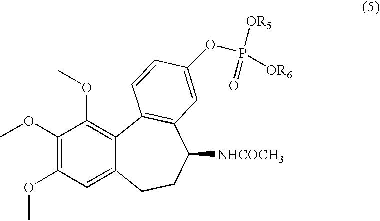

in which each of R5 and R6 is independently selected from the group consisting of H, Li, Na and K, with the proviso that at least one of R5 and R6 is Li, Na or K, said process comprising a step in which a product according to its fifth aspect is converted into a salt with a compound containing an alkali metal cation, said metal cation being chosen from Li, Na and K.

A preferred compound containing an alkali metal cation may be chosen from LiOH, NaOH, and KOH. NaOH is preferred.

According to a seventh aspect, the invention relates to the products obtained by a process in accordance with its sixth aspect.

According to an eighth aspect, the invention relates to pharmaceutical compositions comprising a product according to its fifth or its seventh aspect, in combination with a pharmaceutically acceptable excipient.

According to a ninth aspect, the invention relates to the use of a product according to its fifth or its seventh aspect, for producing a medicinal product which is useful for treating a pathological condition, preferably cancer.

According to a tenth aspect, the invention relates to a product of general formula 1

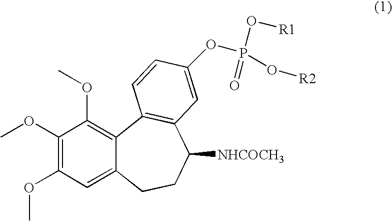

in which

(i) R1 and R2 are, independently, different or identical substituents or else R1 and R2 together form a single substituent;

(ii) R1 and R2 can be cleaved in the presence of at least one transition metal so as to bring about the formation of a phosphate or phosphoric acid group; and

(i) R1 and R2 are halogenated aliphatic groups, or

(ii) R1 and R2 together form a single halogenated aliphatic group.

A preferred halogenated aliphatic group is a hydrocarbon-based chain, for example alkyl, cycloalkyl, comprising at least one halogen selected from the group consisting of chlorine, bromine and iodine.

The hydrocarbon-based chain will advantageously be chosen from those in which the free terminal portion is perhalogenated, preferably from —CH₂—R_{Cl}, R_{Cl} being an aliphatic, linear or cyclic perchlorinated residue.

Very preferred substituents R1 and R2 are each a 2,2,2-trichloroethyl substituent.

Scheme 1 represents a synthetic pathway for the sodium salt of the colchinol phosphate (VI) starting from colchiceine (I), using a process in accordance with the invention.

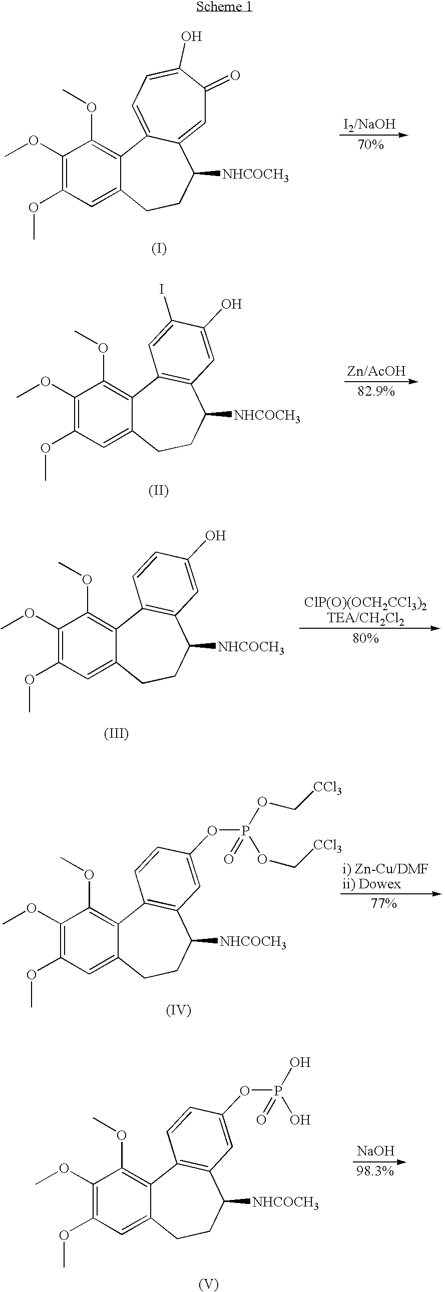

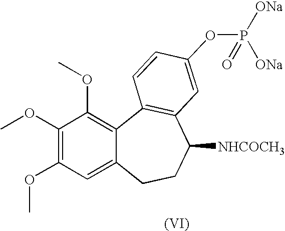

In a first step, colchiceine (I) is reacted with sodium hydroxide in the presence of iodine so as to produce the aromatized iodinated derivative (II) with a 70% yield. The latter is then reduced by reaction with a zinc/acetic acid mixture so as to produce the N-acetylcolchinol (III) with an 82.9% yield.

The phenol function of the N-acetylcolchinol (III) is esterified with a phosphoric acid derivative to produce the compound (IV) with an 80% yield. In a fourth step, the phosphoric ester on the compound (IV) is deprotected with a Zn—Cu amalgam so as to provide the phosphoric acid (V) with a 77% yield, and the latter is then converted into a salt so as to produce the colchinol phosphate (VI), obtained with a 98.3% yield.

The steps shown in Scheme 1 are now described in greater detail by the following examples:

### EXAMPLES

### Product (II): N-[(5S)-3-Hydroxy-2-iodo-9,10,11-trimethoxy-6,7-dihydro-5H-dibenzo[a,c]cyclohepten-5-yl]acetamide

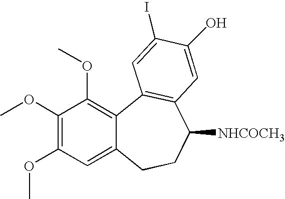

3 l of water, 150 g of NaOH pellets and 150.2 g of colchiceine (I) are placed in a 30 l reactor. The solution obtained is cooled to between 0 and 5° C., and a solution containing 15 l of water, 1.350 kg of Nal and 300 g of I₂ is then added over 1 hour with stirring, while ensuring that the reaction temperature does not exceed 5° C. The orange solution obtained is stirred for 1 hour at between 0 and 5° C., and 250 ml of an aqueous solution of 10% by weight Na₂S₂O₅ are then added. The resulting solution is acidified by adding 185 ml of a concentrated aqueous HCl solution, and 70 ml of aqueous 10% by weight Na₂S₂O₅ solution are then added. The product crystallizes. The solution is stirred for 1 hour at between 0 and 5° C., and the crystals are filtered off, washed with 6 times 125 ml of water and dried under vacuum at 60° C. to obtain 177.7 g (94.5%) of yellow colored crystals; m.p.=210° C.

The entire product obtained above is dissolved in 1.7 l of boiling ethanol, filtered while hot, and then cooled. The product crystallizes spontaneously. The crystals are collected, washed with 70 ml and then 2 times 40 ml of ice-cold ethanol, and then dried under vacuum at 60° C. 131.4 g (74%) of green crystals of the product (II) are collected. m.p. 236° C. Total yield=70%.

### Product (III): N-[(5S)-3-Hydroxy-9,10,11-trimethoxy-6,7-dihydro-5H-dibenzo[a,c]cyclohepten-5-yl]acetamide

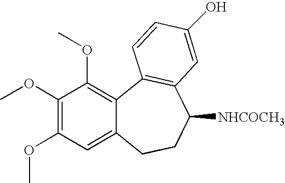

A solution containing 2.6 l of acetic acid and 131 g of product (II) is introduced into a 6 l three-necked flask equipped with a mechanical stirrer, a nitrogen inlet and a coolant. 393 g of powdered zinc are rapidly added to the solution at ambient temperature (18° C). The resulting grey suspension is kept at boiling point for 1 hour and is then cooled to ambient temperature. The solid residue is filtered off and washed with 2 times 175 ml of acetic acid, and the filtrates are collected in a 50 l separator containing 17 l of ice-cold water. The acidic aqueous phase is extracted with 1.5 l and then 3 times 1 l of chloroform. The organic phases are combined, washed with 2 times 1 l of water and dried over Na₂SO₄, and the solvent is then distilled under reduced pressure so as to obtain a residue in the form of an amorphous foam. The latter is dissolved in 200 ml of ethanol and then 400 ml of water are gradually added. The solution becomes cloudy and then crystallizes. The crystals are collected, rinsed with 2 times 40 ml of an ice-cold solution of ethanol/water:½ (vol/vol), and then dried under vacuum at 60° C. 80.3 g (82.9%) of green crystals of product (III) are obtained. m.p.=157° C.

### Product (IV): bis-(2,2,2-Trichloroethyl) (5S)-5-acetylamino-9,10,11-trimethoxy-6,7-dihydro-5H-dibenzo[a,c]cyclohepten-3-ylphosphate

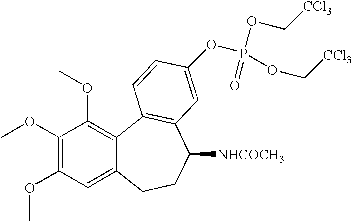

A suspension containing 78.2 g of product (III) in 1.175 l of dichloromethane are introduced into a 4 l three-necked flask equipped with a mechanical stirrer, a nitrogen inlet and a dropping funnel. 61.5 ml of triethylamine are added over a period of 7 minutes at ambient temperature. The mixture becomes brown. The resulting solution is stirred for 20 minutes and a solution containing 166 g of bis-(2,2,2-trichloroethyl) phosphorylchloride (CIP(O)(OCH₂CCl₃)₂) and 400 ml of dichloromethane is then added over a period of 40 minutes. The temperature of the reaction medium is regulated so as not to exceed 28° C. The solution is stirred for 2 hours and is then decomposed by adding 750 ml of water. The organic phase is separated and washed successively with (i) a solution containing 375 ml of water and 375 ml of a saturated NaHCO₃ solution, then with (ii) 750 ml of water. The organic phase is dried over Na₂SO₄, and the solvent is evaporated off under reduced pressure so as to obtain a green resin.

All of the green resin is chromatographed on silica gel using a 7/3 dichloromethane/ethyl acetate mixture to obtain 122.67 g (80%) of a white foam of product (IV).

Analysis: ¹H NMR, 400 MHz, (CD₃)SO; δ (ppm): 1.88 (3H, s); 1.90 (1H, unresolved peak); 2.03 (1H, unresolved peak); 2.18 (1H, unresolved peak); 2.53 (1H, unresolved peak); 3.50 (3H, s); 3.78 (3H, s); 3.84 (3H, s); 4.50 (1H, unresolved peak); 4.95 (4H, unresolved peak); 6.79 (1H, s); 7.27 (2H, unresolved peak); 7.36 (1H, d, J=8.5 Hz); 8.42 (1H, d, J=9.0 Hz).

### Product (V): (5S)-5-Acetylamino-9,10,11-trimethoxy-6,7-dihydro-5H-dibenzo[a,c]cyclo-hepten-3-ylphosphoric acid

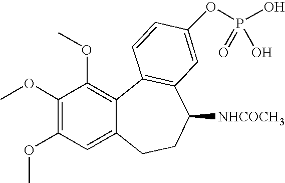

A mixture of 3.065 l of acetic acid and 111.2 g of copper acetate is introduced into a first 6 l three-necked flask equipped with a mechanical stirrer, a nitrogen inlet and a coolant. The suspension is brought to 100° C., and 228.8 g of powdered zinc are then added rapidly with stirring. The heating is maintained for 25 minutes and the suspension is then returned to ambient temperature. The suspension is separated by settling out, the supernatant is taken up by suction under a nitrogen atmosphere, and 1700 ml of acetic acid are introduced into the first three-necked flask. The suspension is stirred and then separated by settling out. The supernatant is taken up by suction. The steps consisting of washing, separating by settling out, and suction are repeated twice with 1 l of DMF. At the end of these two additional washes, 1 l of DMF is introduced into the first three-necked flask and the entire mixture is left under a nitrogen atmosphere.

A solution containing 122.6 g of product (IV) in 1870 ml of DMF is introduced into a second 6 l three-necked flask equipped with a mechanical stirrer, a nitrogen inlet and a coolant. 180.6 g of pentane-1,4-dione and 340 ml of DMF are then added. The suspension prepared in the first three-necked flask is then rapidly added to the mixture, and 240 ml of DMF are then introduced into the second three-necked flask. The reaction medium is heated at 55° C. for 1 hour, and is then cooled to ambient temperature. The residue is filtered and washed with twice 340 ml of DMF, the filtrates are combined and left overnight at ambient temperature, and the solvent is then evaporated off under reduced pressure. The residue is dissolved in a mixture containing 5780 ml of acetonitrile and 1930 ml of water, and 1600 g of Dowex 50WX8 resin, prewashed with 2 l and then twice with 1 l of water, are then added to the solution. The mixture is stirred for approximately 10 minutes and the resin is then filtered off and washed with twice 450 ml of a mixture of acetonitrile/water:3/1 (vol/vol). The filtrate is concentrated under reduced pressure (50 to 60 mbar (50-60 hPa)) at a temperature of between 30 and 35° C. When the acetonitrile is evaporated off, the product crystallizes from the water. The crystals are collected and then dried under vacuum at 40° C. in the presence of CaCl₂ to afford 59.05 g (77%) of white crystals of product (V).

Analysis: ¹H NMR, 400 MHz, (CD₃)SO; δ (ppm): 1.88 (3H, s); 1.88 (1H, unresolved peak); 2.05 (1H, unresolved peak); 2.15 (1H, unresolved peak); 2.51 (1H, unresolved peak); 3.51 (3H, s); 3.78 (3H, s); 3.84 (3H, s); 4.51 (1H, unresolved peak); 6.77 (1H, s); 7.13 (2H, unresolved peak); 7.28 (1H, d, J=8.5 Hz); 8.39 (1H, d, J=9.0 Hz).

### Product (VI): Disodium (5S)-5-acetylamino-9,10,11-trimethoxy-6,7-dihydro-5H-dibenzo[a,c]cyclohepten-3-ylphosphate

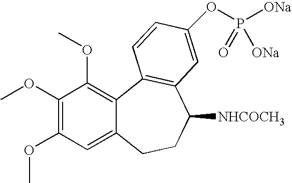

A suspension containing 58.6 g of product (V) obtained above in 600 ml of water is placed in a 2 l three-necked flask equipped with a mechanical stirrer. 263.5 ml of 1 N NaOH are gradually poured into the suspension, taking care not to allow the temperature of the reaction medium to exceed 10° C., until 9≦pH≦10 is obtained. The pale yellow solution obtained is filtered and the water is then evaporated off at 30° C. under 15 to 20 mbar (15-20 hPa), so as to obtain a residue in the form of a yellow resin. The latter is dissolved in 340 ml of ethanol and the product is precipitated by adding 510 ml of diethyl ether. The precipitate is filtered off, washed with twice 170 ml of diethyl ether, and dried under reduced pressure at 40° C. in the presence of CaCl₂ so as to obtain 63.39 g (98.3%) of the expected product (VI) in the form of a white powder.

Analysis: ¹H NMR, 400 MHz, D₂O; δ (ppm): 2.01 (1H, unresolved peak); 2.10 (3H, s); 2.26 (1H, unresolved peak); 2.26 (1H, unresolved peak); 2.54 (1H, unresolved peak); 3.63 (3H, s); 3.89 (6H, s); 4.51 (1H, dd, J=6.0 and 12.0 Hz); 6.85 (1H, s); 7.20 (1H, d, J=2.5 Hz); 7.26 (1H, dd, J=2.5 and 8.5 Hz); 7.35 (1H, d, J=8.5 Hz); HPLC purity: 98.7%

1. A process for preparing a product of general formula 1: 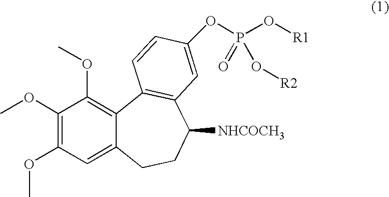
comprising a step of:
coupling a compound of general formula 2: 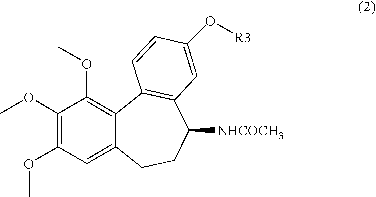
and a compound of general formula 3: : 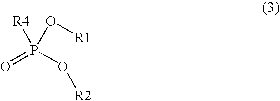
wherein
(i) R1 and R2 are independently selected from the group consisting of alkyl, cycloalkyl, substituted alkyl and substituted cycloalkyl;
(ii) or R1 and R2 together form a single substituent chosen from alkyl, cycloalkyl, substituted alkyl and substituted cycloalkyl; and
(iii) R3 and R4 are labile substituents,
in the presence of a nonaromatic amine a.

2. The process according to claim 1, wherein the nonaromatic amine is a trialkylamine.

3. The process according to claim 2, wherein the trialkylamine is triethylamine.

4. The process according to claim 1, wherein the reaction is carried out in the presence of a halogenated solvent.

5. The process according to claim 4, wherein the halogenated solvent is dichloromethane.

6. The process according to claim 1, wherein
(i) R1 and R2 are halogenated aliphatic groups, or
(ii) R1 and R2 together form a single halogenated aliphatic group.

7. The process according to claim 6, wherein the halogenated aliphatic group is a carbonaceous chain, and in that it comprises at least one halogen selected from the group consisting of chlorine, bromine and iodine.

8. The process according to claim 7, wherein the carbonaceous chain comprises a perhalogenated free terminal portion.

9. The process according to claim 8, wherein the carbonaceous chain comprising a perhalogenated free terminal portion is —CH₂—R_{Cl}, and wherein R_{Cl} is a perchlorinated residue.

10. The process according to claim 9, wherein R1 and R2 are each a 2,2,2-trichloroethyl substituent.

11. The process according to claim 1, wherein R3 is chosen from H, Li, Na and K.

12. The process according to claim 11, wherein R3 is H.

13. The process according to claim 1, wherein R4 is chosen from Cl, Br and I.

14. The process according to claim 13, wherein R4 is Cl.

15. The process according to claim 1, wherein the compound of general formula 1 is bis(2,2,2-trichloroethyl) (5S)-5-acetylamino-9,10,11-trimethoxy-6,7-dihydro-5H-dibenzo[a,c]cyclohepten-3-ylphosphate.

16. The process according to claim 1, wherein the compound of general formula 3 is N-[(5S)-3-hydroxy-9,10,11-trimethoxy-6,7-dihydro-5H-dibenzo[a,c]cyclohepten-5-yl]acetamide, and wherein the compound of general formula 4 is bis(2,2,2-trichloroethyl) phosphorylchloride.

17. The process according to claim 1, wherein the coupling reaction between the compound of general formula 3 and the compound of general formula 4 is carried out at a temperature range from about 0° C. to about 100° C.

18. The process according to claim 17, wherein the coupling reaction is carried out at a temperature range from about 20° C. to about 100° C.

19. The process according to claim 17, wherein the coupling reaction is carried out at a temperature range from about 20° C. to about 50° C.

20. The process for preparing a compound of formula 4: 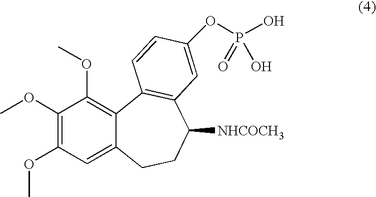
comprising an additional step in which a product obtained by the process according to claim 1 undergoes cleavage of the substituents R1 and R2 in the presence of at least one transition metal.

21. The process according to claim 20, wherein the transition metal is zinc.

22. The process according to claim 20, wherein the substituents R1 and R2 are cleaved in the presence of two different transition metals.

23. The process according to claim 22, wherein the two different transition metals are zinc and copper.

24. The process according to claim 20, further comprising a step for purifying the compound of formula 4 by passing it over ion exchange resin.

25. A process for preparing a compound of general formula 5: 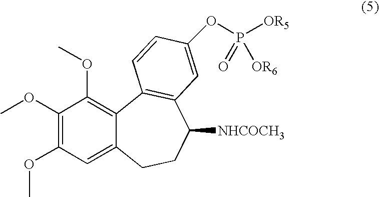
wherein each of R5 and R6 is independently selected from the group consisting of H, Li, Na and K, with the proviso that at least one of R5 and R6 is Li, Na or K,
comprising a step of:
reacting a compound of formula 4: 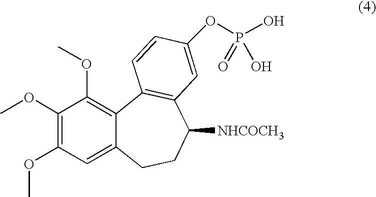
with an alkali compound, wherein the alkali compound is chosen from Li, Na and K.

26. The process according to claim 25, wherein the alkali compound is chosen from LiOH, NaOH, and KOH.

27. The process according to claim 26, wherein the alkali compound is NaOH.

28. A pharmaceutical composition comprising a product obtained by a process according to claim 1, in combination with a pharmaceutically acceptable excipient.

29. A method of treating a pathological condition in a patient comprising administering to said patient a therapeutically effective amount of a compound of formula 1 as set forth in claim 1 or a pharmaceutically acceptable salt thereof optionally in combination with a pharmaceutically acceptable excipient.

30. The method according to claim 29, wherein the pathological condition is cancer.

31. A compound of general formula 1 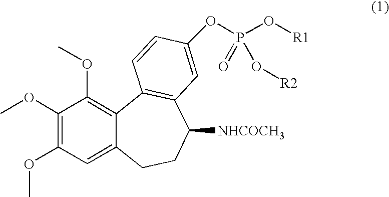
wherein
(i) R1 and R2 are, independently, different or identical substituents or R1 and R2 together form a single substituent;
in that
(ii) R1 and R2 can be cleaved in the presence of at least one transition metal so as to lead to the formation of a phosphate or phosphoric acid group;
and wherein
(i) R1 and R2 are halogenated aliphatic groups, or
(ii) R1 and R2 together form a single halogenated aliphatic group.

32. The compound according to claim 31, wherein the halogenated aliphatic group is a hydrocarbon-based chain, and in that it comprises at least one halogen selected from the group consisting of chlorine, bromine and iodine.

33. The compound according to claim 32, wherein the terminal portion of the halogenated aliphatic group is perhalogenated.

34. The compound according to claim 33, wherein the halogenated aliphatic group is —CH₂—R_{Cl}, and wherein R_{Cl} is a perchlorinated alkyl.

35. The compound according to claim 34, wherein R1 and R2 are each a 2,2,2-trichloroethyl.
